(19) United States
(12) Patent Application Publication
(10) Pub. No.: US 2024/0382346 A1
(43) Pub. Date: Nov. 21, 2024

(54) A MEDICAL DRESSING FOR USE IN CONJUNCTION WITH A MEDICAL DEVICE

(71) Applicant: Mölnlycke Health Care AB, Göteborg (SE)

(72) Inventors: Conny Jakobsson, Lerum (SE); Daniel Melin, Mölnlycke (SE); Christian Wathne, Mölnlycke (SE)

(21) Appl. No.: 18/692,421

(22) PCT Filed: Sep. 28, 2021
(86) PCT No.: PCT/EP2021/076636

§ 371 Date: Mar. 15, 2024

(51) Int. Cl. A61F 13/02; A61F 13/00; A61F 13/0203

(52) U.S. Cl. CPC A61F 13/0259; A61F 13/00059; A61F 13/022

(57) ABSTRACT

Described is a medical dressing for use in conjunction with a medical device. The dressing has an outer layer provided with a plurality of guiding lines indicating how to cut the dressing into a plurality of shaped dressing pieces. The dressing pieces are adapted to fit and to be used in conjunction with a variety of medical devices. Also described is a kit that includes the described medical dressing.

### TECHNICAL FIELD

The present disclosure generally relates to a medical dressing for use in conjunction with a medical device. The dressing comprises an outer layer provided with a plurality of guiding lines indicating how to cut the dressing into a plurality of shaped dressing pieces. The dressing pieces are adapted to fit and to be used in conjunction with a variety of medical devices. The present disclosure also relates to a kit comprising such a medical dressing.

### BACKGROUND

A pressure ulcer is a localized injury to the skin and/or underlying tissue that results from sustained pressure, often in combination with friction and shear. Pressure ulcers often arise among persons being bedridden for various reasons, for instance due to long term hospitalization or other causes of immobility. Areas particularly prone to pressure ulcer formation include bony prominences, such as the heels and the sacrum.

Pressure ulcers may also develop underneath medical devices. Such pressure ulcers are referred to as medical device related pressure ulcers (MDRPU). Medical devices, such as feeding tubes, oxygen delivery devices, intravenous catheters, orthopedic devices, tracheostomy tubes and nasogastric tubes are examples of medical devices commonly used in a hospital or medical setting. These devices are designed to be in continuous and close contact with the body, particularly the facial areas of a patient.

The continuous mechanical loading applied by the medical device, and the rigid materials of such medical devices may abrade the skin and inflict soft tissue distortion, which ultimately may lead to the formation of a pressure ulcer. A medical device related pressure ulcer often mirrors the shape and the location of the medical device.

For example, a common type of equipment utilized in a medical setting is a CPAP mask. The CPAP mask often causes pressure ulcers at the bridge of the nose, the cheeks and on the lower lip. Furthermore, an oxygen tubing often causes pressure ulcers under the nose, the cheeks, and behind the ears of a patient.

Consequently, every patient with a medical device is at risk of developing pressure ulcers, particularly in combination with impaired sensation, poor perfusion, altered tissue tolerance, poor nutrition, edema, and the tendency for moisture to develop under the medical device.

Prevention and management of medical device-related pressure injuries are difficult because the device typically cannot be moved or removed.

To date, the medical personnel may apply various types of tapes and padded material sheets underneath a specific medical device. For example, the medical personnel may utilize various tape pieces and dress the facial area exposed to a medical device. Alternatively, the medical personnel may start from a larger tape or padded material sheet and cut the sheet into a shape that fits with the medical device to be utilized for a specific patient and a specific care scenario.

This is, however, a cumbersome and often difficult procedure. First, because numerous types of medical devices are utilized in a medical setting, yielding a plurality of dermal areas that need to be protected when one or more medical device(s) is/are to be utilized. Furthermore, it is not always clear how the tape or material sheet should be applied or cut to yield optimal protection of the exposed facial areas of a patient. Many times, the face is covered by a puzzle of tape pieces arranged in a random pattern. Furthermore, when starting from a larger material sheet, it may be difficult to understand what dressing shapes are suited for a specific type of medical device or facial part.

Moreover, the application of a medical device to a patient in a hospital or care facility is often associated with a critical care scenario, that may be stressful for the staff. In critical situations, there is often a sense of urgency associated with the application of a medical device to the patient. Hence, there may be limited time available to “dress” the dermal areas of the patient underneath the medical device.

Accordingly, there is a need to provide a simplified and improved means to prevent and manage medical device related pressure ulcers (MDRPU) and to minimize the burden for caregivers dealing with medical device related pressure ulcers, and patients at risk of developing such ulcers.

### SUMMARY

In view of the above-mentioned problems, it is an object of the present disclosure to provide improvements with respect to the prevention of medical device related pressure ulcers, particularly with respect to facilitating the handling and care of such ulcers for staff and patients in a hospital or care facility.

According to a first aspect, there is provided a medical dressing for use in conjunction with a medical device, wherein the dressing comprises an outer layer; the outer layer comprising a folding line dividing the outer layer into a first portion and a second portion; the first and the second portion being symmetric about the folding line, wherein the dressing can be arranged in a first, flat configuration, and in a second, folded configuration about the folding line, wherein the first portion of the outer layer comprises a first set of guiding lines indicating how to cut the dressing in the folded configuration into a first set of dressing pieces, and wherein the second portion comprises a second set of guiding lines indicating how to cut the dressing in the folded configuration into a second set of dressing pieces.

The present disclosure is based on the realization that the provision of a plurality of guiding lines on the outer layer of the dressing greatly facilitates the handling of medical device related pressure ulcers in a hospital or care facility. The staff and caregivers are guided as to how to cut the dressing into a first and a second set of dressing pieces that are tailored to fit and be used in conjunction with a certain type of medical device.

The first and second sets of guiding lines are configured to yield dressing pieces aimed at mitigating pressure points from a variety of medical devices.

The dressing pieces of the first and second sets of dressing pieces have shapes adapted for a variety of curvatures of the facial area. Such curvatures may be particularly prone to the development of pressure ulcers.

The provision of a folding line, about which the dressing is folded when the dressing is cut, allows for a plurality of different guiding lines to fit on a small dressing area. Accordingly, a variety of different dressing shapes may be obtained despite the limited space on the dressing. This allows for an optimized utilization of the dressing material. Less material is needed, and material waste resulting from dressing the patient with various tape pieces in an arbitrary manner or resulting from cutting randomly in larger material or tape sheets, is considerably reduced.

The first set of guiding lines is typically different from the second set of guiding lines.

Accordingly, the first set of dressing pieces is different from the second set of dressing pieces.

The dressing pieces of the first set of dressing pieces may be adapted for a first medical device and the dressing pieces of the second set of dressing pieces may be adapted for a second medical device.

Accordingly, the caregiver may selectively choose to cut in the first or second portion depending on the specific need and specific medical device that is to be applied to the patient. For example, if an oxygen tubing is to be used, the first set of guiding lines on the first portion of the dressing may indicate how to cut the dressing into the dressing shapes adapted for such oxygen tubing. Furthermore, if a CPAP mask or any other medical device is to be used, the second set of guiding lines on the second portion may be adapted for the provision of dressing shapes that fit the CPAP mask (or vice versa).

In urgent and critical situations in a hospital, the staff must typically quickly apply the medical device to the patient. Accordingly, there is a limited time to “dress” the dermal area underneath the medical device. Instead of guessing where and how the tape or material sheet is to be applied, and potentially cutting the tape or material sheet into an incorrect shape, the caregiver is offered “on site” assistance, which allows for the specific requirements and needs of the patient to be met.

Accordingly, this saves time and reduces stress associated with critical care scenarios for the staff dealing with medical devices and pressure ulcers resulting from such devices.

In embodiments, the dressing comprises a backing layer, an adhesive skin contact layer and a release liner configured to be detachably attached to the adhesive skin contact layer, wherein the outer layer comprising the first and the second sets of guiding lines is the backing layer and/or the release liner.

The backing layer and the release liner represent the outermost layers of the dressing, and consequently, the layers viewable by a caregiver.

The release liner is detachably attached to the adhesive skin contact layer and is configured to be removed before the dressing is applied to the skin. If the first and the second sets of guiding lines are provided on the release liner, the dressing is folded and cut into the relevant shapes when the release liner remains attached to the adhesive skin contact layer.

The backing layer is configured to stay attached to the dressing during use and protect the dressing from potential contaminants. The backing layer faces away from the wearer during use.

In embodiments, the first and the second sets of guiding lines are provided on both the backing layer and the release liner.

In embodiments, the outer layer comprising the first and second sets of guiding lines is the release liner and wherein the surface area of the release liner corresponds to the surface area of the adhesive skin contact layer and the backing layer.

Accordingly, the release liner is co-extensive with the adhesive skin contact layer and the backing layer. A release liner being co-extensive in length and width with the adhesive skin contact layer is preferably utilized to optimize the provision of the plurality of guiding lines on the release liner.

A release liner having the same surface area as the backing layer and the adhesive skin contact layer allows for applying the plurality of guiding lines at the peripheral edges of the release liner.

In embodiments, the first set of guiding lines and/or the second set of guiding lines are formed by a print pattern, a weld pattern, a compressed pattern, an embossed pattern, a material deposition pattern, or combinations thereof.

Any pattern that allows the caregiver to visually recognize and follow the pre-applied sets of guiding lines may be utilized.

Typically, the first and the second sets of guiding lines are embossed and/or printed on the outer layer.

The printed guiding lines are easily recognizable on the outer layer, and facilitate cutting into the desirable shaped dressing pieces. The embossed guiding lines may be recognized by touch, and may facilitate cutting of the dressing due to the difference in tactility.

In exemplary embodiments, the first set of guiding lines and/or the second set of guiding lines are printed guiding lines, wherein the printed guiding lines are formed by a colored ink.

The colored ink may have a hydrophobic character to prevent the ink from dissolving in contact with body fluids.

In exemplary embodiments, the first portion of the outer layer has a texture, color or shade that differs from the texture, color or shade of the second portion of the outer layer.

The difference in texture, color and/or shade of the first, and the second portion, respectively, serves to emphasize that the dressing should be folded, and to emphasize that the respective portion yield different dressing shapes tailored for different types of medical devices (depending on which portion the caregiver chooses to cut).

The difference in color or shade may also be regarded as visually appealing to the viewer.

In exemplary embodiments, the first portion of the outer layer is white or transparent and wherein the second portion is colored.

Accordingly, a distinct difference between the portions of the outer layer is provided.

In exemplary embodiments, the colored second portion of the outer layer and the printed guiding lines of the first and the second sets of guiding lines are formed by the same colored ink, wherein the shade of the colored second portion is lighter than the shade of the printed guiding lines.

By utilizing the same colored ink for printing both the second portion and the guiding lines, the manufacturing of the outer layer is significantly facilitated. By varying the contrast and shade of the color of the ink, a distinct and visually appealing contrast is created between the guiding lines and the colored second portion. The shade of the guiding lines may also vary between the guiding lines of the first and second sets of printed guiding lines.

In exemplary embodiments, the outer layer has a surface tension that is higher than the surface tension of the colored ink.

This allows for the printed markings to adhere better to the outer layer. Accordingly, printing is facilitated and the printed markings (guiding lines, colored portions etc.) will have an improved adherence to the outer layer.

In exemplary embodiments, the outer layer comprises a polymeric film, preferably a polyethylene film, a polypropylene film or a polyurethane film.

A polymeric film allows for printing, welding, compressing and/or embossing the markings and guiding lines onto the outer layer.

In exemplary embodiments, the dressing comprises a pad arranged between the backing layer and the adhesive skin contact layer, wherein the pad comprises a pressure-relieving material.

This is beneficial to protect the patient's skin from the load and shear imparted by an overlying medical device. The pad comprises a pressure-relieving material that allows to be cut through.

The dressing of the present disclosure is not restricted to a specific shape, but any shape may be considered. For example, the dressing may be square or rectangular in shape.

Hence, in embodiments, outer layer has a lateral (x) and a longitudinal (y) extension, wherein the folding line is a longitudinal (y) center line; the outer layer being defined by a first and a second longitudinal edge extending in parallel to each other in the lateral (x) direction, and a first and a second lateral edge extending in parallel to each other in the longitudinal (y) direction.

If the dressing is square shaped, the lateral (x) and longitudinal (y) extension of the dressing is the same.

In exemplary embodiments, the first set of guiding lines of the first portion comprises at least a first guiding line extending from a first point on the first longitudinal edge in a curved path to an opposing first point on the second longitudinal edge.

Accordingly, when cutting the first guiding line, three shaped dressing pieces are obtained; i.e. a centrally disposed, substantially hourglass shaped dressing piece, and two respective edge pieces. The substantially hourglass shaped dressing is adapted to fit under the nose of the patient and protect this area when the dressing is used in conjunction with e.g. an oxygen tubing.

The respective edge pieces may be utilized to protect the cheeks of the patient and are preferably arranged to extend from the hourglass shaped dressing towards the ears of the patient. Accordingly, these facial areas are protected from potential scarfing caused by an oxygen tubing.

In exemplary embodiments, the first set of guiding lines of the first portion comprises a second guiding line; the second guiding line extending from a second point on the first longitudinal edge to an opposing second point on the second longitudinal edge; the second guiding line being arranged at a distance from, and generally following the same curved path as the first guiding line.

Accordingly, the respective edge dressing pieces are trimmed at the edges and may fit the cheeks of the patient better.

In exemplary embodiments, the first set of guiding lines of the first portion comprises a third guiding line extending from a first point on the first lateral edge in a curved path to a third point on the second longitudinal edge.

The dressing pieces obtained by cutting the third guiding line may be used to protect the area behind the ears. This area is particularly vulnerable when any type of tubing, e.g. an oxygen tubing is anchored behind the ears of the patient.

In exemplary embodiments, the folding line is a guiding line indicating that the folding line is to be cut.

To maximize the protection of the ears, and to optimize the utilization of the dressing material, the caregiver may, after cutting through the third guiding line, cut the centrally disposed dressing piece along the folding line. Accordingly, four dressing pieces are obtained, two of which are adapted to protect the area behind the ears, and two of which may protect the upper parts of the ears and extending towards the respective cheek of the patient.

Accordingly, the first set of guiding lines provided in the first portion of the outer layer may yield full skin protection when an oxygen tubing is to be applied to a patient.

In exemplary embodiments, the second set of guiding lines of the second portion comprises at least a first guiding line extending from a first point on the second lateral edge, and a second guiding line extending from a second point on the second lateral edge; the first and the second guiding lines being connected at a connecting point in the second portion such that the first and the second guiding lines form the shape of a substantially curved triangle.

Accordingly, when cutting through the first and second guiding lines of the second set of guiding lines, three dressing pieces are obtained; i.e. a centrally disposed large dressing piece, and two substantially curved triangular dressing pieces. The dressing pieces may be utilized to dress the cheeks/and or the chin of the patient. These dressing pieces are configured to mitigate pressure points from e.g. a cervical collar or a tracheostomy tubing.

In exemplary embodiments, the second set of guiding lines of the second portion comprises a third guiding line extending in a curved path from a first point on the folding line to a fourth point of the second longitudinal edge.

Accordingly, an additional substantially triangular dressing piece is obtained as well as a centrally disposed, larger dressing piece having the general shape as a horizontal letter K. The larger K shaped dressing piece may be arranged to cover a portion of the forehead and extend partly over the nose ridge and towards the cheeks in the area circumventing the nose.

The four dressing pieces obtained by means of cutting the first, second and third guiding lines of the second set of guiding lines of the second portion may be utilized to protect the facial areas underneath a CPAP mask.

In exemplary embodiments, the dressing further comprises a fold marking indicating that the dressing should be folded prior to cutting.

Accordingly, the staff or caregivers are directed to folding the dressing prior to cutting the dressing into pieces.

In exemplary embodiments, the release liner is a first release liner, and wherein the dressing further comprises a second release liner arranged between the first release liner and the adhesive skin contact layer, wherein the surface area of the second release liner is smaller than the surface area of the first release liner.

The second release liner is arranged below the first release liner and facilitates the removal of the first release liner.

In exemplary embodiments, the outer layer is the first release liner; the first release liner having a lateral (x) extension defining the width of the first release liner, and a longitudinal (y) extension defining the length of the first release liner; the folding line having a longitudinal (y) extension, wherein the width of the second release liner corresponds to the width of the first release liner, and wherein the length of the second release liner corresponds to from 20 to 60%, preferably from 30 to 40% of the length of the first release liner.

Accordingly, the second release liner extends in a transverse direction with respect to the folding line. This arrangement is beneficial, since after the dressing pieces have been cut into their respective shaped dressing pieces, the majority of the dressing pieces will comprise at least a portion of the second, smaller release liner. Accordingly, the removal of the first release liner overlying the cut-out dressing pieces will be facilitated.

According to a second aspect, there is provided a kit comprising a medical dressing as defined hereinbefore and a medical device.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1A illustrates a top-view of a dressing according to an exemplary embodiment of the present disclosure, wherein the first and second sets of guiding lines are provided on the first release liner.

FIG. 1B is a split-view illustrating a dressing according to an exemplary embodiment of the present disclosure, wherein the first and second sets of guiding lines are provided on the first release liner.

FIG. 1C illustrates the dressing of FIG. 1B in a folded configuration.

FIG. 2A illustrates the shaped dressing pieces formed when the dressing has been cut along the first guiding line of the first set of guiding lines in the folded configuration, according to an exemplary embodiment of the present disclosure.

FIG. 2B illustrates the shaped dressing pieces formed when the dressing has been cut along the first and the second guiding lines of the first set of guiding lines in the folded configuration, according to an exemplary embodiment of the present disclosure.

FIG. 2C illustrates a front-view of a patient wearing the dressing pieces of FIG. 2B.

FIG. 2D illustrates a side-view of a patient wearing the dressing pieces of FIG. 2B.

FIG. 3A illustrates the shaped dressing pieces formed when the dressing has been cut along the third guiding line of the first set of guiding lines in the folded configuration, and along the folding line, according to an exemplary embodiment of the present disclosure

FIGS. 3B and 3C illustrate side views of a patient wearing the dressing pieces of FIG. 3B.

FIG. 4A illustrates the shaped dressing pieces formed when the dressing has been cut along the first and second guiding lines of the second set of guiding lines in the folded configuration, according to an exemplary embodiment of the present disclosure

FIG. 4B illustrates a front-view of a patient wearing the dressing pieces of FIG. 4A.

FIG. 4C illustrates a side-view of a patient wearing the dressing pieces of FIG. 4A.

FIG. 4D illustrates the shaped dressing pieces formed when the dressing has been cut along the first, second and third guiding lines of the second set of guiding lines in the folded configuration, according to an exemplary embodiment of the present disclosure.

FIG. 4E illustrates a front-view of a patient wearing the dressing pieces of FIG. 4D.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

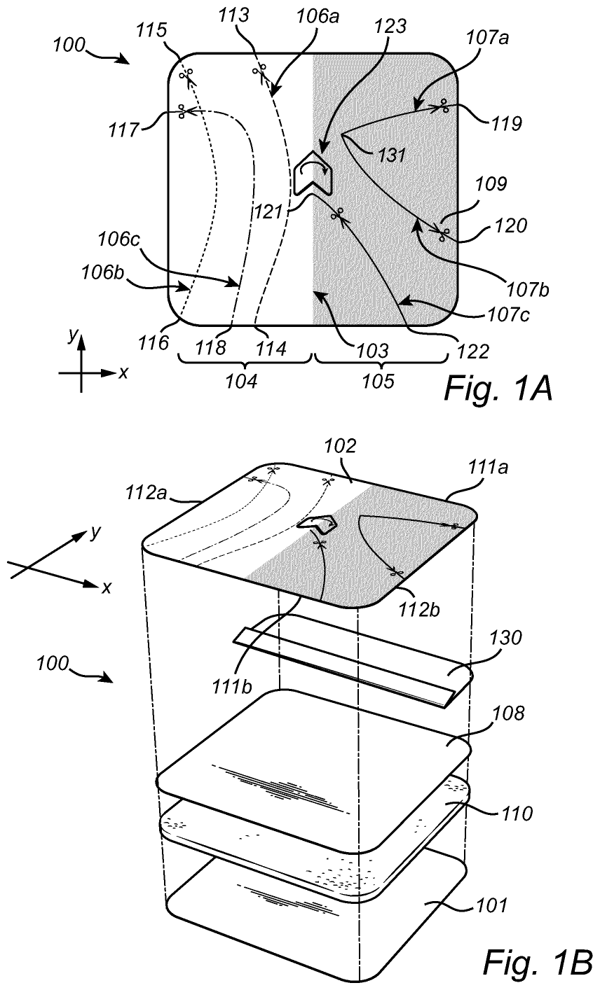

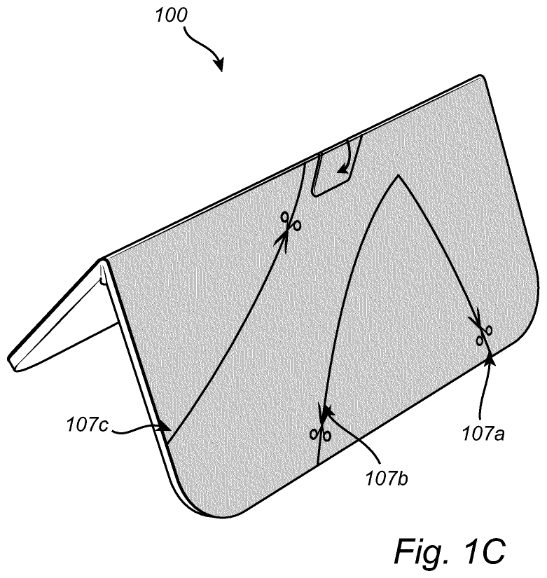

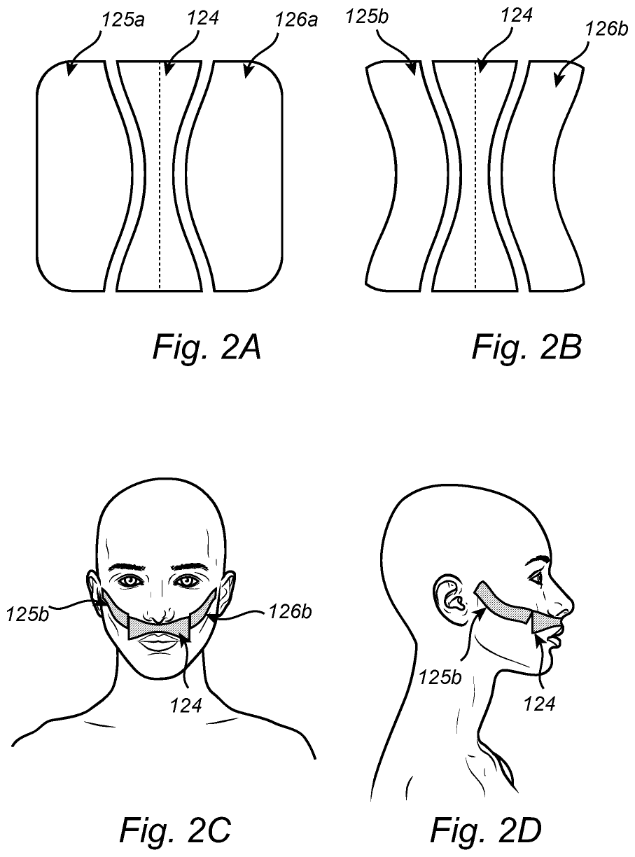

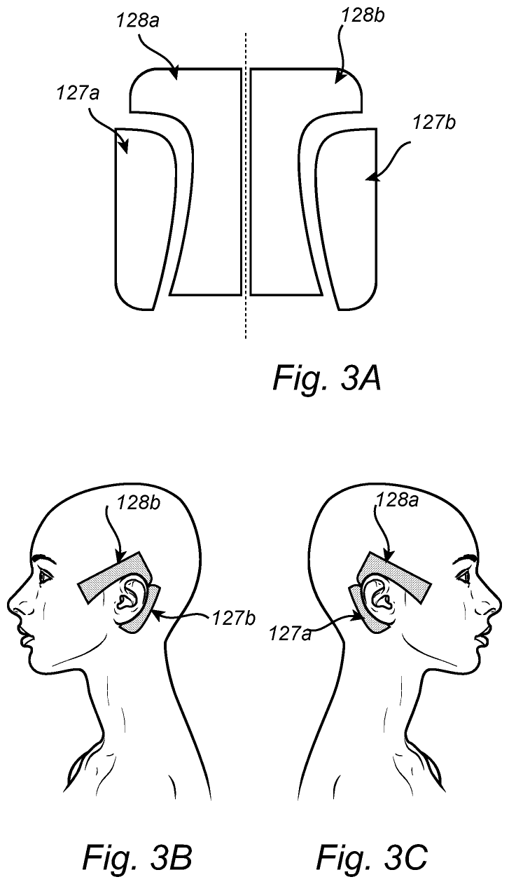

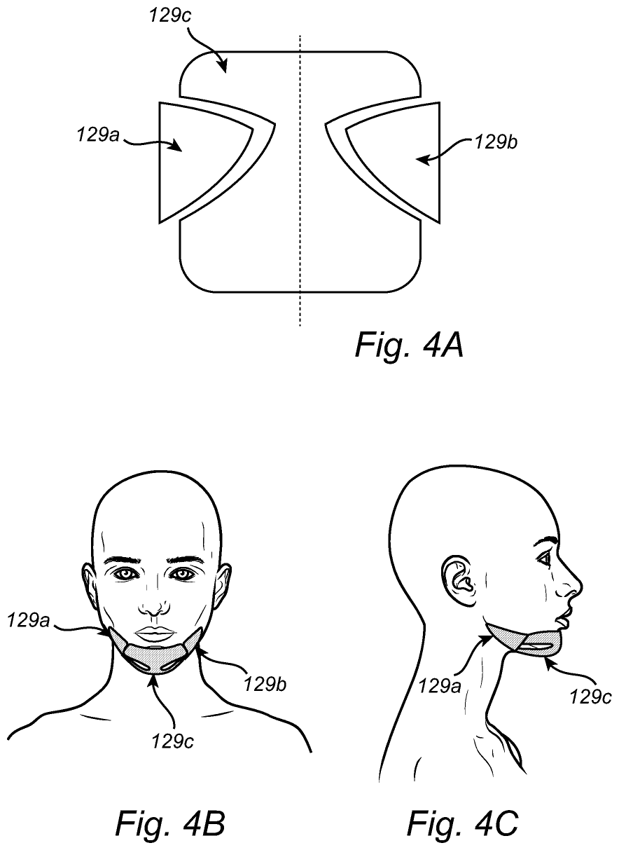

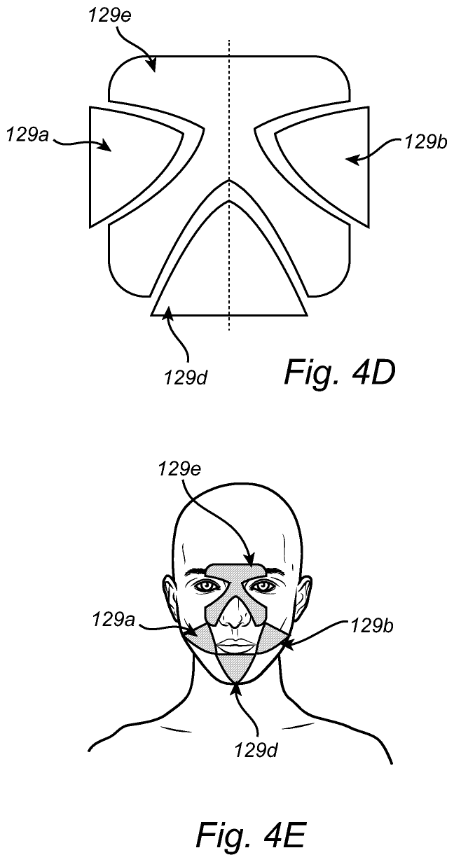

With reference to FIG. 1, a medical dressing 100 for use in conjunction with a medical device according to the present disclosure is conceptually illustrated. The medical dressing comprises an outer layer; the outer layer comprising a folding line 103 dividing the outer layer into a first portion 104 and a second portion 105; the first 104 and the second 105 portion being symmetric about the folding line 103, wherein the dressing 100 can be arranged in a first, flat configuration, and in a second, folded configuration about the folding line 103, wherein the first portion 104 of the outer layer comprises a first set of guiding lines 106a-c indicating how to cut the dressing 100 in the folded configuration into a first set of dressing pieces (see 124,125a-b and 126a-b in FIGS. 2A-D, and 127a-b, 128a-b in FIGS. 3A-C), and wherein the second portion 105 comprises a second set of guiding lines 107a-c indicating how to cut the dressing 100 in the folded configuration into a second set of dressing pieces (see 129a-e in FIGS. 4A-D).

As used herein the term “outer layer” means a layer that faces the caregiver during use, and typically represents the outermost layer of the dressing. The outer layer may be the top layer of the dressing, which in the context of the present disclosure, is referred to as the backing layer. The backing layer faces away from the patient's body in use. Alternatively, the outer layer is the bottom layer, which in the context of the present disclosure, is the release liner. The release liner is detachably attached to the dressing, and is removed prior to application of the dressing to the skin.

As used herein, the term “folding line” means a centrally disposed line dividing the dressing into a first and a second portion, respectively, and which indicates to the caregiver that the dressing should be folded. The folding line may be formed by a print pattern, a weld pattern, a compressed pattern, an embossed pattern, a material deposition pattern, or combinations thereof. The folding line may be a printed and/or embossed folding line. The folding line may also be formed from a difference in texture, color or shade of the first and the second portion of the outer layer. Accordingly, the intersection between the first and the second portion may represent the “folding line” (in embodiments where the texture/color/shade of the first portion is different from the texture/color/shade of the second portion, see FIGS. 1A-1C).

As used herein the term “first and/or second sets of guiding lines” means a plurality of lines that are visually recognizable to the caregiver. The first and/or the second sets of guiding lines may be formed by a print pattern, a weld pattern, a compressed pattern, an embossed pattern, a material deposition pattern, or combinations thereof. Typically, the first and/or second sets of guiding lines are printed or embossed. If the guiding lines are embossed, the guiding lines are also recognizable by touching. If the guiding lines are printed on the outer layer, the printed guiding lines may represent printed solid, continuous, dotted, or discontinuous lines. The printed guiding line may also be associated with scissor markings to further emphasize that the guiding lines should be cut (see 109 in FIG. 1A). Printed guiding lines may be provided on either the backing layer or the release liner. Embossed guiding lines may e.g. utilized in embodiments, where the outer layer is the release liner.

A first or second “set” of guiding lines comprises at least two, e.g. at least three guiding lines.

A first and/or second “set” of dressing pieces means at least three dressing pieces. The dressing pieces may be applied to any dermal area underlying a medical device. Typically, the dressing pieces are applied to a facial area of the patient. The term facial area is to be interpreted broadly. The “facial area” includes all parts of the patient's face, such as the cheeks, nose, nose ridge, forehead, the area between the mouth and the nose, and also includes the chin, neck and the area behind the ears.

As illustrated in FIGS. 1A and 1B, the first set of guiding lines 106a-c is different from the second set of guiding lines 107a-c.

In this context, “different” means that the first set of guiding lines yield differently shaped dressing pieces, than the dressing pieces obtained by the second set of guiding lines, when cut in the folded configuration.

The first set of guiding lines may also be different from the second set of guiding lines in the sense of the character of the guiding lines. As illustrated in FIGS. 1A and 1B, the first set of printed guiding lines (106a-c) comprises discontinuous or dotted lines. The second set of printed guiding lines (107a-c) comprises continuous or bold lines. This is to further emphasize the difference between the first and the second set of guiding lines.

The first set of dressing pieces may be adapted for a first medical device. The second set of dressing pieces may be adapted for a second medical device.

The first and the second sets of dressing pieces may be adapted for the same medical device, but covering different dermal or facial areas. For example, one medical device may require a large number of different dressing pieces, and a variety of dermal areas may require protection. The respective guiding lines of the first and second sets of guiding lines can provide a plurality of different dressing pieces adapted for different dermal areas, but still configured to be used with the same medical device.

Each of the dressing pieces formed by cutting the respective guiding line within the first and the second sets of guiding lines is designed to mitigate at least one pressure point from an overlying medical device.

The guiding lines of the first and second sets of guiding lines are adapted to yield dressing pieces adapted for a particular facial curvature.

The caregiver may decide to cut the first set of guiding lines or the second sets of guiding lines depending on the specific care situation and the specific medical device to be used. A plurality of different dressing shapes may be obtained adapted to fit with one or more specific medical device(s). The guiding lines provided on the outer layer guides the caregiver to first fold the dressing (see FIG. 1C), and then cut the dressing in the folded configuration by following the first, and/or the second set of guiding lines disposed on the outer layer.

The dressing may comprise a backing layer 101, an adhesive skin contact layer 108 and a release liner 102 configured to be detachably attached to the adhesive skin contact layer 108, wherein the outer layer comprising the first (106a-c) and the second (107a-c) sets of guiding lines is the backing layer 101 and/or the release liner 102.

In FIGS. 1A and 1B, the outer layer comprising the first and second sets of guiding lines is the release liner 102. However, it is equally conceivable that the backing layer 101 is provided with the first and second sets of guiding lines.

It is also conceivable to provide the first and second sets of guiding lines on both the backing layer and the release liner.

To facilitate application of the guiding lines (e.g. by means of printing, embossing, welding etc.) onto the release liner, the release liner 102 is preferably co-extensive with the adhesive skin contact layer 108 and with the backing layer 101. This is also beneficial since the dressing is configured to be cut when the co-extensive release liner is attached to the skin contact layer.

Accordingly, the surface area of the release liner 102 corresponds to the surface area of the adhesive skin contact layer 108 and the backing layer 101.

As mentioned, the first set of guiding lines 106a-c and/or the second set of guiding lines 107a-c may be formed by a print pattern, a weld pattern, a compressed pattern, an embossed pattern, a material deposition pattern, or combinations thereof.

In FIGS. 1A-C, the guiding lines (106a-c, 107a-c) are printed on the outer layer; i.e. the release liner 102.

The guiding lines may alternatively, or additionally, be embossed. The embossed lines may facilitate cutting of the dressing due to the difference in tactility.

The guiding lines may be printed on the outer layer by any conventional printing technique known in the art, including, but not limited to a gravure printing, a flexographic printing, an offset printing, an ink jet printing and the like. Preferably, a flexographic printing technique is utilized.

The release liner 102 has first side facing the interior of the dressing, and an opposing second side, facing the viewer. The guiding lines may be printed on either side of the release liner 102, as long as they are visibly recognizable. Typically, the guiding lines are printed on the second side of the release liner.

In embodiments where the backing layer is the outer layer comprising the printed first and second sets of guiding lines, the printed guiding lines are typically provided on the first side of the backing layer; i.e. the side facing the interior of the dressing. Accordingly, the printed markings do not directly contact the medical device, and potential removal or transfer of the ink is therefore prevented.

The printed guiding lines are preferably formed by a colored ink.

Any colored ink may be used as long as it remains permanently adhered to the outer layer. Preferably, a flexographic ink is used. The ink may be water based or solvent based; i.e. the ink may comprise an organic solvent, such as an alcohol, ester etc. that can dissolve the pigment, resin and potentially other additives.

In embodiments where the sets of guiding lines are embossed, the embossed guiding lines may be provided by conventional embossing techniques using e.g. thermal bond, ultrasonic bond and/or pressure. For example, the outer layer may be arranged between two rolls, wherein one of the rolls is engraved with the visual pattern and the other is flat.

The first portion 104 of the outer layer may have a texture, color or shade that differs from the texture, color or shade of the second portion 105 of the outer layer.

In FIGS. 1A-C, the first portion 104 is white or transparent and the second portion 105 is colored.

The colored second portion 105 of the outer layer and the printed guiding lines of the first set of guiding lines (106a-c) and the second set of guiding lines (107a-c) may be formed by the same colored ink, wherein the shade of the colored second portion is lighter than the shade of the printed guiding lines.

The shade or color of the second portion 105 is not limited to a particular shade or color, but is lighter or brighter in color or shade than the color/shade of the first and second sets of guiding lines (in cases where these are formed by a print pattern).

The difference in color and/or shade of the first, and the second portion distinguishes the respective sets of guiding lines from one another, and the caregiver may choose to cut from the colored or the transparent/white portion depending on the desired dressing shapes and medical device to be utilized. The difference in color and/or shade also emphasizes that the dressing should be folded prior to cutting.

Preferably, the ink has a color that contrast with the color of the release liner or the backing layer. For example, the color of the ink may be grey, black, purple, pink, blue, green, red and any shades thereof.

Preferably, the color of the ink is PMS 254 (Pantone Matching System).

The difference in the shade between the second portion and the printed guiding lines may be from 40 to 80%, e.g. from 50 to 70%.

The “difference in shade” may e.g. be a difference in opacity of the colored ink, a difference in the degree of saturation of the colored ink or a difference in raster density of the colored ink.

The outer layer (101,102) may have a surface tension that is higher than the surface tension of the colored ink.

This allows for an improved adherence of the colored ink to the outer layer to be printed. The surface tension of the outer layer may be increased by subjecting the outer layer to corona treatment.

The outer layer; i.e. the backing layer and/or the release liner typically comprises a polymeric film, preferably a polyethylene film, a polypropylene film or a polyurethane film.

The outer layer may be formed by the polymeric film or a laminate comprising the polymeric film. The thickness of the polymeric film is typically in the range of from 10 to 140 μm, e.g. from 20 to 100 μm.

The release liner preferably comprises a polyethylene or a polypropylene film having a thickness in the range of from 40 to 140 μm, e.g. from 70 to 100 μm.

The backing layer preferably comprises a polyurethane film having a thickness in the range of from 10 to 50 μm, e.g. from 15 to 40 μm.

The backing layer is preferably thinner than the release liner since this layer should remain relatively flexible during use.

As illustrated in FIG. 1B, the dressing 100 comprises a pad 110 arranged between the backing layer 101 and the adhesive skin contact layer 108, wherein the pad 110 comprises a pressure-relieving material.

Accordingly, the dressing provides for an improved pressure-relieving effect and prevents scarfing caused by the medical device.

The pad is not limited to a specific material as long as it allows for the dressing to be cut through and yields a pressure-relieving effect.

The pad is preferably absorbent. This is to improve the handling of body fluids, e.g. blood, pus or sweat.

For example, the pad may comprise a hydrophilic and/or absorbent foam. Preferably, the pad comprises a polyurethane foam.

The pad 110 may comprise one or more layers. If the pad comprises a plurality of pad-forming layers, the pad-forming layers may be laminated or attached to each other to facilitate cutting through the dressing. Preferably, the pad is void of superabsorbent particles or gel forming fibers, which may “leak” from the dressing during cutting.

The thickness of the pad may be in the range of from 1 to 5 mm, e.g. from 2-3 mm. If the thickness of the pad is to large, it may become difficult to fold and/or cut through the dressing.

The pad 110 is typically co-extensive with the backing layer 101 and the adhesive skin contact layer 108.

As used herein, the term “adhesive skin contact layer” means a layer configured to detachably adhere the dressing to a dermal surface. In other words, the adhesive skin contact layer is configured to contact the skin of a wearer.

Preferably, the adhesive skin contact layer comprises a silicone-based adhesive. A silicone-based adhesive is gentle to the skin and may be removed from the skin without causing any trauma. For example, the adhesive skin contact layer may comprise a silicone gel. The silicone gel may be provided as a coating on the pad.

The adhesive skin contact layer may comprise one or more sub-layers. For example, the adhesive skin contact layer may comprise a polymeric film and an adhesive silicone gel layer, wherein the adhesive silicone gel layer is arranged to contact the skin.

In embodiments where the dressing comprises a pad, the backing layer 101 may be adhesively attached to the pad. Alternatively, the backing layer 101 may be laminated to the pad. For example, heat lamination may be utilized to apply the backing layer 101 to the pad.

As best illustrated in FIG. 1B, the outer layer has a lateral (x) and a longitudinal (y) extension; the folding line 103 having a longitudinal (y) extension; and wherein the outer layer is defined by a first longitudinal edge 111a and a second longitudinal edge 111b; the first 111a and the second 111b longitudinal edges extending in parallel to each other in the lateral (x) direction, and a first lateral edge 112a and an a second lateral edge 112b; the first 112a and the second 112b lateral edges extending in parallel to each other in the longitudinal (y) direction.

The dressings in FIGS. 1A-1C are square shaped. Consequently, the lateral (x) extension and the longitudinal (y) extension are the same. Also, each lateral and longitudinal edge have the same length. If the dressing has a rectangular shape, the lateral (x) extension is larger than the longitudinal (y) extension (or vice versa).

The “longitudinal (y) extension” corresponds to the length direction of the outer layer (or the dressing).

The “lateral (x) extension” corresponds to the width direction of the outer layer (or the dressing).

The medical dressing of the present disclosure is by no means limited to a specific shape, but any shape may be conceivable.

The medical dressing may have a width and/or length in the range of from 8 to 25 cm, preferably from 10 to 20 cm, and most preferably from 12 to 15 cm.

As illustrated in FIG. 1, the first set of guiding lines 106a-c of the first portion 104 comprises at least a first guiding line 106a extending from a first point 113 on the first longitudinal edge 111a in a curved path to an opposing first point 114 on the second longitudinal edge 111b.

The curved path forms a convex curve between the first point 113 on the first longitudinal edge 111a and the opposing first point 114 on the second longitudinal edge 111b. The convex curve is arranged to bulge towards the folding line 103.

When cutting the first guiding line, the dressing pieces (124, 125a, 126a) illustrated in FIG. 2A are obtained. Accordingly, a centrally disposed, substantially hourglass shaped dressing piece 124 and two respective edge pieces (125a, 126a) are provided.

The hourglass shaped dressing 124 has a narrow waste portion which improves the conformability of the dressing when applied under the nose of a patient.

The central point of the convex curve formed by the first guiding line 106a is preferably arranged at a distance of from 3 to 12 mm, e.g from 5 to 10 mm from the folding line. This is to yield an hourglass shaped dressing having a waste portion particularly suited for placement under the nose.

The first set of guiding lines 106a-c of the first portion 104 may comprise a second guiding line 106b; the second guiding line 106b extending from a second point 115 on the first longitudinal edge 111a to an opposing second point 116 on the second longitudinal edge 111b; the second guiding line 106b being arranged at a distance from, and generally following the same curved path as the first guiding line 106a.

The curved path forms a convex curve between a second point 115 on the first longitudinal edge 111a to an opposing second point 116 on the second longitudinal edge.

The convex curve is arranged to bulge towards the folding line 103 and generally follows the same curved path as the first guiding line 106a.

The first 115 and second 116 points of the second guiding line 106b are arranged closer to the first lateral edge 112a than the first 113 and the second 114 points of the first guiding line 106a.

When cutting the second guiding line 106b, the dressing shapes (125b, 126b) illustrated in FIG. 2B are formed. These shapes resemble the shapes of FIG. 2A, but are trimmed at the edges.

The dressing shapes formed by cutting the first guiding line 106a, and optionally the second guiding line 106b are adapted to fit under e.g. an oxygen tube, and serve to protect the cheeks and the area beneath the nose as illustrated in FIGS. 2C and 2D.

The hourglass shaped dressing 124 may be arranged beneath the nose, and the respective edge piece (125a, 126a) may be arranged adjacent to the hourglass shaped dressing 124 and extend across the cheeks towards the ears (as illustrated in FIG. 2D).

As illustrated in FIG. 1A, the first set of guiding lines 106a-c of the first portion 104 comprises a third guiding line 106c extending from a first point 117 on the first lateral edge 112a in a curved path to a third point 118 on the second longitudinal edge 111b.

The third point 118 on the second longitudinal edge 111b is arranged between the first point 114 from which the first guiding line extends, and the second point 116 from which the second guiding line 106b extends.

As can be seen in FIG. 1A, the third guiding line forms a slightly convex curve between the respective end-points (118 and 117). The third guiding line 106c intersects the second guiding line 106b.

The outer layer may be divided by a lateral (x) center line into an upper portion and a lower portion. The first point 117, from which the third guiding line 106 extends is arranged in the upper portion of the outer layer.

When cutting the third guiding line 106c, the dressing pieces 127a-b in FIG. 3A are formed. These shapes may e.g. be utilized to protect the area behind the ears (see FIGS. 3B and 3C).

The folding line 103 may be a guiding line indicating that the folding line 103 is to be cut

As illustrated in FIG. 3A, when cutting through the folding line 103, four dressing shaped pieces (127a-b, 128a-b) are obtained.

The dressing pieces resulting from cutting the folding line 103 may be arranged to cover the upper area of the ear and extend towards the cheek and/or forehead of the patient (see FIGS. 3B and 3C).

The dressing pieces obtained by cutting the first set of guiding lines and as illustrated in FIGS. 2A-D and 3A-3C offer full protection of the skin when e.g. an oxygen tubing is to be applied to a patient.

As illustrated in FIGS. 1A and 1B, the second set of guiding lines 107a-c of the second portion 105 comprises at least a first guiding line 107a extending from a first point 119 on the second lateral edge 112b of the second portion 105, and a second guiding line 107b extending from a second point 120 on the second lateral edge 112b of the second portion 105; the first 107a and the second 107b guiding lines being connected at a connecting point 131 in the second portion 105 such that the first 107a and the second 107b guiding lines form the shape of a substantially curved triangle.

Accordingly, three dressing pieces (129a-c) that are suitable to dress the cheeks or the chin of the patient are obtained (see FIGS. 4B and 4C). Such an arrangement is beneficial for use in conjunction with a cervical collar or a tracheostomy tubing.

The outer layer may be divided by a lateral (x) center line into an upper portion and a lower portion. The first point 119 on the second lateral edge 112b, from which the first guiding line 107a extends, is arranged in the upper portion. The second point 120 on the second lateral edge 112b, from which the second guiding line 107b extends is arranged in the lower portion.

The connecting point 131 connecting the first 107a and second guiding line 107b in the second portion is preferably arranged in the upper portion of the outer layer. The connecting point 131 may e.g. be provided at a distance of from 6 to 13 mm, e.g. from 8 to 10 mm from the folding line 103. This is to improve the fit to the bridge of the nose of a patient.

The second set of guiding lines 107a-c of the second portion 105 may comprise a third guiding line 107c extending in a curved path from a first point 121 on the folding line 103 to a fourth point 122 of the second longitudinal edge 111b.

The outer layer may be divided by a lateral (x) center line into an upper portion and a lower portion. The first point 121 of the folding line 103 is arranged in the lower portion; i.e. below a central point of the folding line. If the outer layer comprises a centrally disposed fold marking 123, the first point 121 of the folding line is arranged adjacent to the fold marking, but does not coincide therewith.

Accordingly, when the dressing is cut in the folded configuration, a substantially curved triangle is provided, similar in shape to that defined by the first and the second guiding lines, explained hereinbefore.

The dressing pieces (129a-b, 129d-e) illustrated in FIG. 4D are thereby obtained. The centrally disposed dressing piece 129e has the general shape of a horizontal letter K and is larger than the remaining dressing pieces (129a-b, 129d). The dressing pieces may be used to dress a patient in accordance with FIG. 4E. In FIG. 4E, the dressing piece 129e is arranged to cover a portion of the forehead and extending partly over the nose bridge and the areas adjacent to the nose. The substantially triangular dressing pieces (129a, 129b, 129d) are arranged to cover the cheeks and the chin in the area circumventing the mouth of a patient.

Such an arrangement is suitable for use in conjunction with a CPAP mask.

As illustrated in FIGS. 1A-C, the respective guiding lines are associated with scissor markings 109, to further emphasize that the guiding lines should be cut.

As illustrated in FIGS. 1A-1B, the outer layer 101 may further comprise a fold marking 123 indicating that the dressing should be folded prior to cutting.

The fold marking may be formed by a print pattern, a weld pattern, a compressed pattern, an embossed pattern, a material deposition pattern, or combinations thereof.

Typically, the fold marking 123 is a printed marking.

The fold marking 123 is not limited to a specific shape, but any marking that guides the caregiver to fold the dressing may be utilized. The fold marking 123 may be arranged in a central position on the folding line 103.

In FIG. 1A-1B, the fold marking 123 comprises a curved arrow, which serves emphasize that the dressing should be folded.

In embodiments where the fold marking 123 is a printed marking, the same ink may be utilized as that used for printing the first and/or second sets of guiding lines.

As illustrated in FIG. 1B, the dressing may further comprise a second release liner 130 arranged between the first release liner 102 and the adhesive skin contact layer 108, wherein the surface area of the second release liner 130 is smaller than the surface area of the first release liner 102.

The provision of the second release liner 130 facilitates removal of the first release liner 102 from the adhesive skin contact layer 108.

The outer layer is, in FIG. 1B the first release liner 102; the first release liner 102 having a lateral (x) extension defining the width of the first release liner 102, and a longitudinal (y) extension defining the length of the first release liner 102; the folding line 103 having a longitudinal (y) extension, wherein the width of the second release liner 130 corresponds to the width of the first release liner 102, and wherein the length of the second release liner 130 corresponds to from 20 to 60%, preferably from 30 to 40% of the length of the first release liner 102.

Accordingly, the second release liner extends in a transverse direction with respect to the folding line. This arrangement is beneficial, since after the dressing pieces have been cut into their respective shaped dressing pieces, the majority of the dressing pieces will comprise at least a portion of the second, smaller release liner. Accordingly, the removal of the first release liner overlying the cut-out dressing pieces will be facilitated.

As illustrated in FIG. 1B, the second release liner 130 extends between the first 112a and second 112b lateral edges and is arranged to cover at least a portion of the first 106a, second 106b and third 106c guiding lines of the first set of guiding lines, as well as the first guiding line 107a of the second set of guiding lines. Accordingly, all of the dressing pieces formed by these guiding lines will comprise a part of the second release liner such that removal of the first release liner 102 is facilitated. The dressing piece formed by the third guiding line 107c of the second set of guiding lines will only be covered by the first release liner portion.

The width of the first release liner is measured from the first lateral edge 112a to the second lateral edge 112b. The length of the first release liner is measured from the first longitudinal edge 111a to the second longitudinal edge 111b.

The second release liner 130 may be configured to cover from 20 to 50%, e.g. from 30 to 40% of the surface area of the adhesive skin contact layer 108. The first release liner 102 may be configured to cover from 50 to 80% e.g. from 60 to 70% of the surface are of the adhesive skin contact layer 108. Accordingly, the area of the first release liner 102 overlapping the second release liner 104 is from 20 to 50%, e.g. from 30 to 40%.

The second release liner 130 is typically folded (see FIG. 1B). The folded portion forms a gripping tab that the applicator can grasp after the first release liner has been removed.

In embodiments, the first release liner 102 and the second release liner 130 are formed from different materials. For example, the first release liner 102 may comprise a first polymeric film, and the second release liner 130 may comprise a second polymeric film, wherein the first polymeric film is different from the second polymeric film.

The inventors have found that this is particularly advantageous if the first release liner is co-extensive with the adhesive skin contact layer and the backing layer. During manufacturing of the dressing, and particularly during assembly of the dressing components, the first and the second release liners tend to adhere to each other, particularly at the interface between the first and the second release liner, which, in the present case, is relatively large. One reason for such undesired adherence may be that heat generated from friction during assembly of the dressing components can cause the release liners to become partially welded or fused together. Hence, different materials with different melting points are advantageously utilized for the first and the second release liners. This facilitates the removal of the first release liner from the underlying second release liner (and from the dressing).

Suitably, the first polymeric film is a polyethylene film and the second polymeric film is a polypropylene film.

These polymeric films have different melting points and undesired fusion between the first and the second release liner is prevented during assembly or manufacturing of the medical dressing

In another aspect, the present disclosure relates to a kit comprising a medical dressing as described hereinbefore and at least one medical device

Terms, definitions and embodiments of all aspects of the present disclosure apply mutatis mutandis to the other aspects of the present disclosure.

Even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A medical dressing for use in conjunction with a medical device, wherein said dressing comprises an outer layer: said outer layer comprising a folding line dividing said outer layer into a first portion and a second portion; said first and said second portion being symmetric about said folding line, wherein said dressing can be arranged in a first, flat configuration, and in a second, folded configuration about said folding line, wherein said first portion of said outer layer comprises a first set of guiding lines indicating how to cut said dressing in said folded configuration into a first set of dressing pieces, and wherein said second portion comprises a second set of guiding lines indicating how to cut said dressing in said folded configuration into a second set of dressing pieces.

2. The medical dressing according to claim 1, wherein said first set of guiding lines is different from said second set of guiding lines.

3. The medical dressing according to claim 1, wherein the dressing pieces of said first set of dressing pieces are adapted for a first medical device and wherein the dressing pieces of said second set of dressing pieces are adapted for a second medical device.

4. The medical dressing according to claim 1, wherein said dressing comprises a backing layer, an adhesive skin contact layer and a release liner configured to be detachably attached to said adhesive skin contact layer, wherein said outer layer comprising said first and said second sets of guiding lines is said backing layer and/or said release liner.

5. The medical dressing according to claim 4, wherein said outer layer comprising said first and said second sets of guiding lines is said release liner and wherein the surface area of said release liner corresponds to the surface area of said adhesive skin contact layer and said backing layer.

6. The medical dressing according to claim 1, wherein said first set of guiding lines and/or said second set of guiding lines are formed by a print pattern, a weld pattern, a compressed pattern, an embossed pattern, a material deposition pattern, or combinations thereof.

7. The medical dressing according to claim 1, wherein the guiding lines of said first and second sets of guiding lines are printed guiding lines formed by a colored ink.

8. (canceled)

9. (canceled)

10. (canceled)

11. The medical dressing according to claim 7, wherein said outer layer has a surface tension that is higher than the surface tension of said colored ink.

12. The medical dressing according to claim 1, wherein said outer layer is said backing layer and/or said release liner and wherein said outer layer comprises a polymeric film.

13. The medical dressing according to claim 2, wherein said dressing comprises a pad arranged between said backing layer and said adhesive skin contact layer, wherein said pad comprises a pressure-relieving material.

14. The medical dressing according to claim 1, wherein said outer layer has a lateral (x) and a longitudinal (y) extension, wherein said folding line is a longitudinal (y) center line: said outer layer being defined by a first and a second longitudinal edge extending in parallel to each other in the lateral (x) direction, and a first and an a second lateral edge extending in parallel to each other in the longitudinal (y) direction.

15. The medical dressing according to claim 14, wherein said first set of guiding lines of said first portion comprises at least a first guiding line extending from a first point on said first longitudinal edge in a curved path to an opposing first point on said second longitudinal edge.

16. The medical dressing according to claim 15, wherein said first set of guiding lines of said first portion comprises a second guiding line; said second guiding line extending from a second point on said first longitudinal edge to an opposing second point on said second longitudinal edge; said second guiding line being arranged at a distance from, and generally following the same curved path as said first guiding line.

17. The medical dressing according to claim 14, wherein said first set of guiding lines of said first portion comprises a third guiding line extending from a first point on said first lateral edge in a curved path to a third point on said second longitudinal edge.

18. The medical dressing according to claim to claim 1, wherein said folding line is a guiding line indicating that said folding line is to be cut.

19. The medical dressing according to claim 14, wherein said second set of guiding lines of said second portion comprises at least a first guiding line extending from a first point on said second lateral edge, and a second guiding line extending from a second point on said second lateral edge; said first and said second guiding lines being connected at a connecting point in said second portion such that said first and said second guiding lines form the shape of a substantially curved triangle.

20. The medical dressing according to claim 14, wherein said second set of guiding lines of said second portion comprises a third guiding line extending in a curved path from a first point on said folding line to a fourth point on said second longitudinal edge.

21. The medical dressing according to claim 1, wherein said dressing further comprises a fold marking indicating that the dressing should be folded prior to cutting.

22. The medical dressing according to claim 1, wherein said release liner is a first release liner, and wherein said dressing further comprises a second release liner arranged between said first release liner and said adhesive skin contact layer, wherein the surface area of said second release liner is smaller than the surface area of said first release liner.

23. (canceled)

24. A kit comprising a medical dressing according to claim 1 and at least one medical device.
